(19) United States
(12) Patent Application Publication
(10) Pub. No.: US 2005/0142169 A1
(43) Pub. Date: Jun. 30, 2005

(54) Palatability enhanced composition and method for animal consumption

(76) Inventors: Gilbert I. Imafidon, Lawrence, KS (US); Chi-Kuen Shu, Topeka, KS (US); Pavinee Chinachoti, Topeka, KS (US)

Correspondence Address: HARNESS, DICKEY & PIERCE, P.L.C., 7700 BONHOMME, SUITE 400, ST LOUIS, MO, 63105, US

(21) Appl. No.: 11/001,425

(22) Filed: Dec. 1, 2004

Related U.S. Application Data
(60) Provisional application No. 60/526,013, filed on Dec. 1, 2003.
(60) Provisional application No. 60/526,013, filed on Dec. 1, 2003.

(51) Int. Cl. A23K 1/165; A23K 1/17; A61K 31/35; A61K 31/365; A61K 31/12; A01K 29/00

(52) U.S. Cl. 424/442; 514/690; 514/472; 514/460; 426/465; 119/710

(57) ABSTRACT

This invention is directed generally to compositions (including foods, supplements, treats, toys, etc.) for animal consumption, particularly compositions comprising one or more alpha cyclic enolone compound(s). This invention also is directed generally to methods for using such compositions. This invention is further directed generally to processes for making such compositions.

### PRIORITY CLAIM TO RELATED PATENT APPLICATION

This patent claims priority to U.S. Provisional Patent Application Ser. No. 60/526,013 (filed Dec. 1, 2003). The entire text of U.S. Provisional Patent Application Ser. No. 60/526,013 is incorporated herein by reference.

### FIELD OF THE INVENTION

This invention is directed generally to compositions (including foods, supplements, treats, toys, etc.) for animal consumption, particularly compositions exhibiting enhanced palatability, and particularly compositions that comprise an alpha cyclic enolone compound. This invention is further directed generally to processes for making such compositions. This invention also is directed generally to methods for using such compositions.

### BACKGROUND OF THE INVENTION

Pets are well taken care of by their owners with a proper selection of foods. Those foods can be and include their usual diet, supplements, snacks, treats and toys. Pets, like humans, are attracted to and eat more regularly foods which they find palatable. Therefore, palatability enhancers are an extremely important addition to compositions for animal consumption.

Numerous potential palatability enhancers are available for pets, the only limitation appearing to be the imagination of the selector. Some are commercially available as flavoring agents. Others, inter alia, can be prepared by family recipes handed down over generations, and others are continually being synthesized anew, extracted from natural products, or digested from various animal organs. The success of these materials in compositions for animal consumption is not predictable to any great extent. Firstly, a potential palatability enhancer should be compatible with the composition during processing and packaging and it should possess a practical extended shelf-life. Secondly, the palatability enhancer should be appealing to the animals' sense of taste, smell, physical attractiveness and other attributes which appear in the overall composition. Thirdly, the palatability enhancer should be compatible with the animal after ingestion such that it does not cause any significant problems to the animal, particularly gastrointestinal problems.

### SUMMARY OF THE INVENTION

Briefly, therefore, this invention is directed, in part, to a composition for animal consumption, such as, for example, a food, nutritional supplement, treat, or toy. The composition comprises a palatability enhancing amount of one or more alpha cyclic enolone compound(s).

In one contemplated embodiment, the composition comprises a palatability enhancing amount of a 2-hydroxy-2-cyclopenten-1-one compound of formula I:

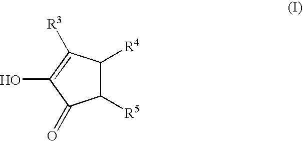

wherein each of R³, R⁴ and R⁵ are independently selected from the group consisting of hydrogen and alkyl.

In another contemplated embodiment, the composition comprises a palatability enhancing amount of a 4-hydroxy-3(2H)-furanone compound of formula II:

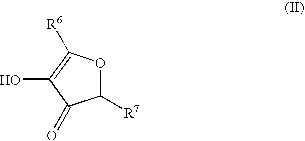

wherein each of R⁶ and R⁷ are independently selected from the group consisting of hydrogen and alkyl.

In another contemplated embodiment, the composition comprises a palatability enhancing amount of a 3-hydroxy-2(5H)-furanone compound of formula (III):

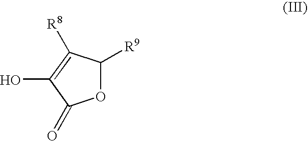

wherein each of R⁸ and R⁹ are independently selected from the group consisting of hydrogen and alkyl.

In still another contemplated embodiment, the composition comprises a palatability enhancing amount of a 3-hydroxy-4-pyranone compound of formula IV:

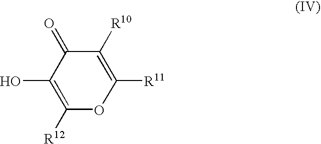

wherein each of R¹⁰, R¹¹ and R¹² are independently selected from the group consisting of hydrogen and alkyl.

This invention also is directed to an animal treat that comprises one or more alpha cyclic enolone compound(s).

This invention also is directed to an animal toy that comprises one or more alpha cyclic enolone compound(s).

This invention also is directed to processes for preparing such compositions, treats, and toys and methods for using such compositions, treats and toys.

Further benefits of Applicants' invention will be apparent to one skilled in the art from reading this patent.

### DETAILED DESCRIPTION OF THE PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

It has been found in accordance with this invention that alpha cyclic enolone compounds can be suitable palatability enhancing agents for use in compositions for animal consumption. For example, without being held to a particular theory, Applicants have found that inclusion of one or more alpha cyclic enolone compounds into an composition for animal consumption imparts enhanced palatability when measured against standard animal food compositions.

Generally, alpha cyclic enolone compounds contain carbonyl, alpha-enolic hydroxyl and a short alkyl groups on contiguous carbon atoms in a five- or six-membered carbocyclic or O-heterocyclic ring. Alpha cyclic enolone compounds possess pleasant caramel/sweet aroma and have been used as food flavors for human consumption. Alpha cyclic enolone compounds may be further defined by four general classes including 2-hydroxy-2-cyclopenten-1-one compounds, 4-hydroxy-3(2H)-furanone compounds, 3-hydroxy-2(5H)-furanone compounds and 3-hydroxy-4-pyranone compounds.

Thus, in a contemplated embodiment, the alpha cyclic enolone compound comprises a 2-hydroxy-2-cyclopenten-1-one compound of formula I:

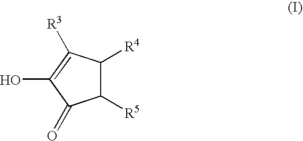

wherein each of R³, R⁴ and R⁵ are independently selected from the group consisting of hydrogen and alkyl. Examples of suitable 2-hydroxy-2-cyclopenten-1-one compounds for use in the present invention include without limitation 3-methyl-2-hydroxy-2-cyclopenten-1-one, 3-ethyl-2-hydroxy-2-cyclopenten-1-one, 3,5-dimethyl-2-hydroxy-2-cyclopenten-1-one, 3,4-dimethyl-2-hydroxy-2-cyclopenten-1-one, 3-ethyl-4-methyl-2-hydroxy-2-cyclopenten-1-one and homologs thereof.

In another contemplated embodiment, the alpha cyclic enolone compound comprises a 4-hydroxy-3(2H)-furanone compound of the formula II:

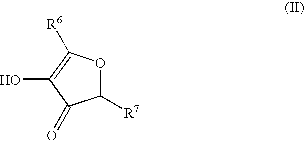

wherein each of R⁶ and R⁷ are independently selected from the group consisting of hydrogen and alkyl. Examples of suitable 4-hydroxy-3(2H)-furanone compounds for use in the present invention include without limitation 5-methyl-4-hydroxy-3(2H)-furanone, 2,5-dimethyl-4-hydroxy-3(2H)-furanone, 5-methyl-2-ethyl-4-hydroxy-3(2H)-furanone, 5-ethyl-2-methyl-4-hydroxy-3(2H)-furanone, 2,5-diethyl-4-hydroxy-3(2H)-furanone and homologs thereof.

In another contemplated embodiment, the alpha cyclic enolone comprises a 3-hydroxy-2(5H)-furanone compound of formula (III):

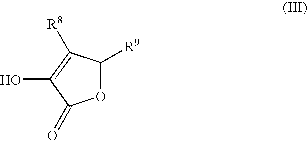

wherein each of R⁸ and R⁹ are independently selected from the group consisting of hydrogen and alkyl. Examples of suitable 3-hydroxy-2(5H)-furanone compounds for use in the present invention include without limitation 4,5-dimethyl-3-hydroxy-2(5H)-furanone, 4-ethyl-5-methyl-3-hydroxy-2(5H)-furanone and homologs thereof.

In another contemplated embodiment, the alpha cyclic enolone compound comprises a 3-hydroxy-4-pyranone compound of formula IV: 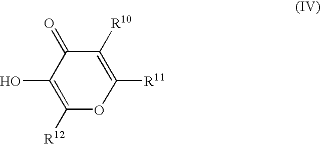
wherein each of R¹⁰, R¹¹ and R¹² are independently selected from the group consisting of hydrogen and alkyl. Examples of suitable 3-hydroxy-4-pyranone compounds for use in the present invention include without limitation 2-methyl-3-hydroxy-4-pyranone, 2-ethyl-3-hydroxy-4-pyranone and homologs thereof.

In a certain preferred embodiment, the alpha cyclic enolone compound is 2,5-dimethyl-4-hydroxy-3(2H)-furanone or 5-methyl-2-ethyl-4-hydroxy-3(2H)-furanone. 2,5-dimethyl-4-hydroxy-3(2H)-furanone is also commercially available under the trade name FURANEOL from Firmenich Int'l SA of Geneva, Switzerland. Further, it is important to note that mixtures of one or more alpha cyclic enolone compound is contemplated as suitable for use in the present invention.

It is contemplated that the compositions and methods of this invention may be useful for a variety of mammals, including non-human mammals such as non-human primates (e.g., monkeys, chimpanzees, etc.), companion animals (e.g., dogs, cats, equine, etc.), farm animals (e.g., goats, sheep, swine, bovine, etc.), laboratory animals (e.g., mice, rats, etc.), and wild and zoo animals (e.g., wolves, bears, deer, etc.).

In some embodiments of this invention, for example, the animal is a monogastric mammal (i.e., a mammal having a single stomach), such as, for example, a non-human primate, dog, cat, rabbit, horse, or swine.

In other embodiments of this invention, the animal is a carnivorous mammal, i.e., a meat-eating mammal.

In other embodiments of this invention, the animal is an omnivorous mammal, i.e., a mammal that eats both plants and meat.

In other embodiments of this invention, the animal is a companion animal.

In other embodiments of this invention, the animal is a cat.

In other embodiments of this invention, the animal is a dog.

In other embodiments of this invention, the animal is a rabbit.

In other embodiments of this invention, the animal is a swine.

In other embodiments of this invention, the animal is a horse.

This invention contemplates a variety of compositions containing one or more alpha cyclic enolone compound. Contemplated compositions include, for example, foods, supplements, treats, and toys (typically chewable and consumable toys).

The alpha cyclic enolone compound(s) preferably is present in the composition in an amount which provides enhanced palatability to the composition. Typically, such a palatability enhancing amount may be as little as 0.0001% (i.e., about 1 ppm) by weight of the composition on a dry matter basis to any amount which maintains enhanced palatability without any adverse reactions.

In a certain preferred embodiment for feline consumption, the amount of alpha cyclic enolone compound(s) in the composition typically comprises at least about 0.0001% (i.e., about 1 ppm) or about 0.001% (i.e., about 10 ppm) or about 0.0015% (i.e., about 15 ppm) by weight of the food on a dry matter basis to about 0.2% (i.e., about 2000 ppm) or about 0.5% (i.e., about 5000 ppm) or about 1.0% (i.e., about 10,000 ppm). Likewise, in a certain preferred embodiment for canine consumption, the amount of alpha cyclic enolone compound(s) in the composition typically comprises at least about 0.0001% (i.e., about 1 ppm) or about 0.001% (i.e., about 10 ppm) or about 0.005% (i.e., about 50 ppm) or about 0.006% (i.e., about 60 ppm) or about 0.0075% (i.e., about 75 ppm) by weight of the food on a dry matter basis to about 0.2% (i.e., about 2000 ppm) or about 0.5% (i.e., about 5000 ppm) or about 1.0% (i.e., about 10,000 ppm).

The alpha cyclic enolone compound and other ingredients preferably are present at concentrations that are not deleterious to the intended animal's health. Thus, for example, the alpha cyclic enolone compound and other ingredients preferably are present at concentrations that do not cause undesirable effects on digestion, particularly long term undesirable effects on digestion, such as undesirable effects lasting several days or longer. Undesirable effects on digestion may include, for example, constipation or diarrhea.

In some embodiments, the composition is a food. Although both liquid and solid foods are contemplated, solid foods are typically preferred. Where the food is solid, the alpha cyclic enolone compound may be coated on the food, incorporated into the food, or both. Contemplated foods include both dry foods or wet foods.

In a contemplated embodiment, the composition is a food that comprises the following:
(a) at least about 0.0001% to about 2% of an alpha cyclic enolone compound; and
(b) at least one of the following:
   (i) from about 5% to about 70% (or from about 10% to about 70%, or from about 10% to about 60%) protein, and
   (ii) from about 2% to about 50% (or from about 5% to about 50%, or from about 5% to about 40%) fat.

In such an embodiment, it is contemplated that the composition also may, for example, comprise at least one of the following:.
(a) no greater than about 50% (or from about 5% to about 45%) carbohydrate,
(b) no greater than about 40% (or from about 1% to about 20%, or from about 1% to about 5.5%) dietary fiber, and
(c) no greater than about 15% (or no greater than about 10%, or from about 2% to about 8%) of one or more nutritional balancing agents.

In another contemplated embodiment, the composition is a food that comprises the following:
(a) at least about 0.0001% to about 2% of an alpha cyclic enolone compound,
(b) from about 5% to about 70% (or from about 10% to about 70%, or from about 10% to about 60%) protein,
(c) from about 2% to about 50% (or from about 5% to about 50%, or from about 5% to about 40%) fat,
(d) no greater than about 50% (or from about 5% to about 45%) carbohydrate,
(e) no greater than about 40% (or from about 1% to about 20%, or from about 1% to about 5.5%) dietary fiber, and
(f) no greater than about 15% (or no greater than about 10%, or from about 2% to about 8%) of one or more nutritional balancing agents.

Specific preferred amounts for each component in a composition will depend on a variety of factors including, for example, the species of animal consuming the composition; the particular components included in the composition; the age, weight, general health, sex, and diet of the animal; the animal's consumption rate; the type of composition condition(s) being treated; and the like. Thus, the component amounts may vary widely, and may even deviate from the preferred proportions set forth in this patent.

It is contemplated that the protein in the compositions of the present invention may be supplied by a variety sources, including, plant sources, animals sources, or both. Animal sources include, for example, meat, meat by-products, dairy, eggs, etc. Meats include, for example, the flesh of poultry; fish; and mammals (e.g., cattle, swine, sheep, goats, and the like). Meat by-products include, for example, lungs, kidneys, brain, livers, and stomachs and intestines (preferably freed of essentially all or all their contents).

The fat and carbohydrate in the compositions of the present invention may be supplied by a variety of sources, including, for example, meat, meat by-products, other animal or plant protein sources, grains, and mixtures thereof. Grains include, for example, wheat, corn, barley, and rice.

Fiber in the compositions of the present invention may be supplied from a variety of sources, including, for example, vegetable fiber sources such as cellulose, beet pulp, peanut hulls, and soy fiber.

Particularly in instances when the composition is an animal's food, vitamins and minerals preferably are included in amounts required to avoid deficiency and maintain health. These amounts are readily available in the art. The National Research Council (NRC), for example, provides recommended amounts of such ingredients for farm animals. See, e.g., Nutrient Requirements of Swine (10th Rev. Ed., Nat'l Academy Press, Wash. D.C., 1998), Nutrient Requirements of Poultry (9th Rev. Ed., Nat'l Academy Press, Wash. D.C., 1994), Nutrient Requirements of Horses (5th Rev. Ed., Nat'l Academy Press, Wash. D.C., 1989), etc. And the American Feed Control Officials (AAFCO), for example, provides recommended amounts of such ingredients for dogs and cats. See American Feed Control Officials, Incorp., Official publication, pp. 126-140 (2003). Contemplated vitamins generally useful as food additives include, for example, vitamin A, vitamin B1, vitamin B2, vitamin B6, vitamin B12, vitamin C, vitamin D, vitamin E, vitamin H (biotin), vitamin K, folic acid, inositol, niacin, and pantothenic acid. Contemplated minerals and trace elements generally useful as food additives include, for example, calcium, phosphorus, sodium, potassium, magnesium, copper, zinc, chorine, and iron salts.

The compositions of the present invention may further contain additives known in the art. Preferably, such additives are present in amounts that do not impair the purpose and effect provided by the invention. Examples of contemplated additives include, for example, substances with a stabilizing effect, organoleptic substances, processing aids and substances that provide nutritional benefits.

Contemplated stabilizing substances include, for example, substances that tend to increase the shelf life of the composition. Potentially suitable examples of such substances include, for example, preservatives, antioxidants, synergists and sequestrants, packaging gases, stabilizers, emulsifiers, thickeners, gelling agents, and humectants. Examples of emulsifiers and/or thickening agents include, for example, gelatin, cellulose ethers, starch, starch esters, starch ethers, and modified starches.

Contemplated additives for coloring, palatability, and nutritional purposes include, for example, colorants; iron oxide, sodium chloride, potassium citrate, potassium chloride, and other edible salts; vitamins; minerals; and flavoring. The amount of such additives in a composition typically is up to 5% (dry basis of the composition).

Supplements include, for example, a feed used with another feed to improve the nutritive balance or performance of the total. Contemplated supplements include compositions that are fed undiluted as a supplement to other feeds, offered free choice with other parts of an animal's ration that are separately available, or diluted and mixed with an animal's regular feed to produce a complete feed. The AAFCO, for example, provides a discussion relating to supplements in the American Feed Control Officials, Incorp. Official Publication, p. 220 (2003). Supplements may be in various forms including, for example, powders, liquids, syrups, pills, encapsulated compositions, etc.

Treats include, for example, compositions that are given to an animal to entice the animal to eat during a non-meal time. Contemplated treats for canines include, for example, dog bones. Treats may be nutritional, wherein the composition comprises one or more nutrients, and may, for example, have a composition as described above for food. Non-nutritional treats encompass any other treats that are non-toxic. The an alpha cyclic enolone compound can be coated onto the treat, incorporated into the treat, or both.

Toys include, for example, chewable toys. Contemplated toys for dogs include, for example, artificial bones. The alpha cyclic enolone compound can form a coating on the surface of the toy or on the surface of a component of the toy, be incorporated partially or fully throughout the toy, or both. In a contemplated embodiment, the an alpha cyclic enolone compound is orally accessible by the intended user. There a wide range of suitable toys currently marketed. See, e.g.,, U.S. Pat. No. 5,339,771 (and references disclosed in U.S. Pat. No. 5,339,771). See also, e.g., U.S. Pat. No. 5,419,283 (and references disclosed in U.S. Pat. No. 5,419,283). It should be recognized that this invention contemplates both partially consumable toys (e.g. toys comprising plastic components) and fully consumable toys (e.g., rawhides and various artificial bones). It should be further recognized that this invention contemplates toys for both human and non-human use, particularly for companion, farm, and zoo animal use, and particularly for dog, cat, or bird use.

In preparing a composition of the present invention, the components of the composition are adjusted so that the alpha cyclic enolone compound is present in the composition at a concentration of at least about 1 parts per million (ppm) (i.e., about 0.0001%) to about 20,000 ppm (i.e., about 2.0%) based on the dry content of the composition. The alpha cyclic enolone compound may, for example, be incorporated into the composition during the processing of the formulation, such as during and/or after mixing of other components of the composition. Distribution of these components into the composition can be accomplished by conventional means.

Compositions of the present invention (particularly foods) can be prepared in a dry form using conventional processes. In one contemplated embodiment, dry ingredients, including, for example, animal protein sources, plant protein sources, grains, etc. are ground and mixed together. Moist or liquid ingredients, including fats, oils, animal protein sources, water, etc. are then added to and mixed with the dry mix. The mixture is then processed into kibbles or similar dry pieces. Kibble is often formed using an extrusion process in which the mixture of dry and wet ingredients is subjected to mechanical work at a high pressure and temperature, and forced through small openings and cut off into kibble by a rotating knife. The wet kibble is then dried and optionally coated with one or more topical coatings which may include, for example, flavors, fats, oils, powders, and the like. Kibble also can be made from the dough using a baking process, rather than extrusion, wherein the dough is placed into a mold before dry-heat processing.

The palatability enhancing alpha cyclic enolone compound(s) may be added to the food composition in its normal preparation procedure such as mixing, extrusion, baking and the like or is preferably added after its preparation, for example, post extrusion, such as by spraying or coating the surface of the food. This is particularly desirable for dry foods wherein the extruded strands can be contacted with the alpha cyclic enolone compound (or a solution comprising the alpha cyclic enolone compound) by spraying or coating the extruded strands before the strands are cut into a kibble, or the kibble can be contacted with the alpha cyclic enolone compound (or a solution comprising the alpha cyclic enolone compound) by spraying, coating or dipping the kibble per se.

For topical application to a food, the compound(s) may be mixed with a carrier composition to facilitate application to the surface of the food composition. For example, a liquid, slurry, light gel, or watery solid can all be utilized as a carrier for the compound(s) of this composition. Standard spraying or dipping apparatus can be employed to apply the compound(s) to the surface of the food composition. An example of such a carrier is a minced animal by-product treated with proteases in conjunction with amino acids, reducing sugar(s) and thiamin. The carrier is then mixed with the alpha cyclic enolone compound and coated onto a kibble, thereby preparing a very palatable and acceptable dry food. In a certain preferred embodiment, the alpha cyclic enolone compound may simply be mixed with a commercial liquid palatant enhancer or other flavor composition to create a novel flavor palatant which can then be topically applied to the composition. Suitable commercial liquid palatant enhancers for use with the alpha cyclic enolone compounds in the present invention include any known or commercially available liquid palatant enhancers commercially available from pet food palatant enhancer or other flavor suppliers known to those of skill in the art.

Compositions of the present invention (particularly foods) can be prepared in a canned or wet form using conventional pet food processes. In one contemplated embodiment, ground animal (e.g., mammal, poultry, and/or fish) proteinaceous tissues are mixed with the other ingredients, including fish oils, cereal grains, other nutritionally balancing ingredients, special purpose additives (e.g., vitamin and mineral mixtures, inorganic salts, cellulose and beet pulp, bulking agents, and the like); and water that sufficient for processing is also added. These ingredients preferably are mixed in a vessel suitable for heating while blending the components. Heating of the mixture may be effected using any suitable manner, such as, for example, by direct steam injection or by using a vessel fitted with a heat exchanger. Following the addition of the last ingredient, the mixture is heated to a temperature range of from about 50° F. to about 212° F. Temperatures outside this range are acceptable, but may be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. This is usually accomplished by heating to temperatures of greater than about 230° F. for an appropriate time, which is dependent on, for example, the temperature used and the composition.

For wet foods, the alpha cyclic enolone compound can be incorporated into the wet food composition along with a carrier, preferably an alcohol composition such as propylene glycol or dipropylene glycol, a cyclodextrin, a maltodextrin or a starch. Alternatively, the alpha cyclic enolone compound can be mixed into the dry materials prior to forming the wet food composition.

Treats of the present invention can be prepared by, for example, an extrusion or baking process similar to those described above for dry food. Other processes also may be used to either coat the flavoring composition on the exterior of existing treat forms, or inject it into an existing treat form.

Animal toys of the present invention are typically prepared by coating any existing toy with the flavoring composition, for example, the alpha cyclic enolone compound mixed with a carrier composition.

### EXAMPLES

The following examples are merely illustrative, and not limiting to this disclosure in any way.

### Example 1

This example demonstrates the effect of 2,5-dimethyl-4-hydroxy-3(2H)-furanone as a palatability enhancer in a dry, commercial dog food composition. The experiment comprised mixing various amounts of 2,5-dimethyl-4-hydroxy-3(2H)-furanone (0.2% to 1.2%) with a commercial liquid palatant enhancer as a carrier to form a flavored palatant (hereinafter test flavor). The test flavor (2.0%, dry weight basis) was topically applied to a dry, commercial dog food (Hill's Canine Maintenance Adult, a Science Diet for adult dogs commercially available from Hill's Pet Nutrition, Inc., Topeka, Kans.) after sequential application of soy bean oil (1.2%, dry weight basis) and choice white grease (7.9%, dry weight basis) to form a test composition.

Each test composition was compared against a control composition in a palatability test. The control compositions were prepared by topically applying the commercial liquid palatant enhance (2.0% dry weight basis) to the dry, commercial dog food (Hill's Canine Maintenance Adult) after sequential application of soy bean oil (1.2%, dry weight basis) and choice white grease (7.9%, dry weight basis). Palatability was determined by comparing the test composition and the control composition in a standard two-bowl preference test over two days with 20 dogs. As shown below in Table 1, each of the test compositions exhibited significant palatability enhancement over the control compositions on the basis of intake and/or preference. No evidence of any intolerance of the test palatant was observed in the dogs following intake.

TABLE 1
Results for Example 1

| | 2,5-dimethyl-4- | | | | |
|---|---|---|---|---|---|
| | hydroxy- | | | | |
| | 3(2H)-furanone | | | | |
| | in liquid palatant | | | | Statistical |
| Exp. | enhancer (ppm, | Status | Intake | Preference | Signifi- |
| No. | dry wt. of kibble) | (vs. control) | Ratio | (%) | cance |
| 1 | 0.0 | Parity | 0.5751 | 55.0/40.0 | 0.0953 |
| 2 | 43.5 | Parity | 0.5705 | 61.1/27.8 | 0.1510 |
| 3 | 87.1 | Win | 0.7361 | 90.0/10.0 | 0.0001 |
| 4 | 130.5 | Win | 0.5673 | 50.0/25.0 | 0.0269 |
| 5 | 217.5 | Win | 0.6575 | 60.0/40.0 | 0.0046 |
| 6 | 217.5 | Win | 0.8206 | 95.0/5.0 | 0.0001 |

### Example 2

This example demonstrates the effect of 2,5-dimethyl-4-hydroxy-3(2H)-furanone as a palatability enhancer for a dry, commercial dog food composition. The experiment comprised mixing various amounts of 2,5-dimethyl-4-hydroxy-3(2H)-furanone with a commercial liquid palatant enhancer as a carrier to form a test flavor. Each of the test flavors (2.0%, dry weight basis) was topically applied to a dry, commercial dog food (Canine r/d from Hill's Pet Nutrition, Inc.) after sequential application of soy bean oil (2.0%, dry weight basis) and choice white grease (0.4%, dry weight basis) to form a test composition.

Each test composition was compared against a control composition in a palatability test as described in Example 1. The control compositions were prepared by topically applying the commercial liquid palatant enhancer (2.0% dry weight basis) to the dry, commercial dog food (Canine r/d) after sequential application of soy bean oil (2.0%, dry weight basis) and choice white grease (0.4%, dry weight basis). Palatability was determined by comparing the test composition and the control composition in a standard two-bowl preference test over two days with 20 dogs as described in Example 1. Results are shown in Table 2 below.

TABLE 2
Results for Example 2.

| | 2,5-dimethyl-4- | | | | |
|---|---|---|---|---|---|
| | hydroxy- | | | | |
| | 3(2H)-furanone | | | | |
| | in liquid palatant | | | | Statistical |
| Exp. | enhancer (ppm, | Status | Intake | Preference | Signifi- |
| No. | dry wt. of kibble) | (vs. control) | Ratio | (%) | cance |
| 1 | 21.75 | Parity | 0.5843 | 50.0/42.9 | 0.1317 |
| 2 | 43.5 | Win | 0.6667 | 77.8/16.7 | 0.0005 |
| 3 | 87.1 | Parity | 0.5732 | 64.7/29.4 | 0.0675 |
| 4 | 174.0 | Parity | 0.5523 | 50.0/31.3 | 0.0934 |
| 5 | 217.5 | Parity | 0.5047 | 33.3/66.7 | 0.4762 |
| 6 | 282.8 | Parity | 0.4297 | 52.9/47.1 | 0.1192 |

### Example 3

This example demonstrates the effect of 2,5-dimethyl-4-hydroxy-3(2H)-furanone as a palatability enhancer for a dry, commercial dog food composition. The experiment comprised mixing various amounts of 2,5-dimethyl-4-hydroxy-3(2H)-furanone with a commercial liquid palatant enhancer as a carrier to form a test flavor. Each of the test flavors (3.5%, dry weight basis) was topically applied to a dry, commercial dog food (Canine w/d from Hill's Pet Nutrition, Inc.) after topical application of a commercial dry palatant enhancer in choice white grease (0.6%, dry weight basis) to form a test composition.

Each test composition was compared against a control composition in a palatability test as described in Example 1. The control compositions were prepared by topically applying the commercial liquid palatant enhancer (3.5% dry weight basis) to the dry, commercial dog food (Canine w/d) after application of a commercial dry palatant enhancer in choice white grease (0.6%, dry weight basis). Palatability was determined by comparing the test composition and the control composition in a standard two-bowl preference test over two days with 20 dogs as described in Example 1. Results are shown in Table 3 below.

TABLE 3
Results for Example 3.

| | 2,5-dimethyl-4- | | | | |
|---|---|---|---|---|---|
| | hydroxy- | | | | |
| | 3(2H)-furanone | | | | |
| | in liquid palatant | | | | Statistical |
| Exp. | enhancer (ppm, | Status | Intake | Preference | Signifi- |
| No. | dry wt. of kibble) | (vs. control) | Ratio | (%) | cance |
| 1 | 87.1 | Win | 0.7301 | 83.3/16.7 | 0.0001 |
| 2 | 130.5 | Win | 0.7323 | 88.0/8.0 | 0.0001 |
| 3 | 174.0 | Win | 0.6669 | 76.0/24.0 | 0.0008 |
| 4 | 217.5 | Parity | 0.5713 | 60.0/40.0 | 0.0754 |

### Example 4

This example demonstrates the effect of 2,5-dimethyl-4-hydroxy-3(2H)-furanone as a palatability enhancer for a dry, commercial dog food composition. The experiment comprised mixing various amounts of 2,5-dimethyl-4-hydroxy-3(2H)-furanone with a commercial liquid palatant enhancer as a carrier to form a test flavor. Each of the test flavors (2.4%, dry weight basis) was topically applied to a dry, commercial dog food (Canine w/d from Hill's Pet Nutrition, Inc.) after application of a commercial dry palatant enhancer in choice white grease (0.6%, dry weight basis) to form a test composition.

Each test composition was compared against a control composition in a palatability test as described in Example 1. The control compositions were prepared by topically applying the commercial liquid palatant enhancer (2.4% dry weight basis) to the dry, commercial dog food (Canine w/d) after application of a commercial dry palatant enhancer in choice white grease (0.6%, dry weight basis). Palatability was determined by comparing the test composition and the control composition in a standard two-bowl preference test over two days with 20 dogs as described in Example 1. Results are shown in Table 4 below.

TABLE 4
Results for Example 4.

| | 2,5-dimethyl-4- | | | | |
|---|---|---|---|---|---|
| | hydroxy- | | | | |
| | 3(2H)-furanone | | | | |
| | in liquid palatant | | | | Statistical |
| Exp. | enhancer (ppm, | Status | Intake | Preference | Signifi- |
| No. | dry wt. of kibble) | (vs. control) | Ratio | (%) | cance |
| 1 | 87.1 | Win | 0.6939 | 76.0/16.0 | 0.0008 |
| 2 | 130.5 | Win | 0.6616 | 72.0/28.0 | 0.0069 |
| 3 | 174.0 | Win | 0.6947 | 76.0/24.0 | 0.0020 |
| 4 | 217.5 | Win | 0.7773 | 92.0/8.0 | 0.0001 |
| 5 | 282.8 | Win | 0.8079 | 87.5/8.3 | 0.0001 |

Second set of experiments

| | | | | | |
|---|---|---|---|---|---|
| 6 | 87.1 | Win | 0.6058 | 60.0/40.0 | 0.0094 |
| 7 | 130.5 | Win | 0.6857 | 76.0/24.0 | 0.0011 |
| 8 | 174.0 | Parity | 0.5568 | 56.0/40.0 | 0.1160 |
| 9 | 217.5 | Parity | 0.5453 | 60.0/40.0 | 0.1421 |
| 10 | 282.8 | Win | 0.7429 | 84.0/16.0 | 0.0001 |

### Example 5

This example demonstrates the effect of 2,5-dimethyl-4-hydroxy-3(2H)-furanone as a palatability enhancer for a dry, commercial cat food composition. The experiment comprised mixing various amounts of 2,5-dimethyl-4-hydroxy-3(2H)-one furanone with a commercial liquid palatant enhancer as a carrier to form a test flavor. Each of the test flavors (0.5%, dry weight basis) was topically applied to a dry, commercial cat food (Feline w/d from Hill's Pet Nutrition, Inc.) after sequential application of water (1.5%), soy bean oil (1.7%, dry weight basis) and choice white grease (1.0%, dry weight basis). Finally, a commercial dry palatant enhancer (1.4% dry weight basis) was topically applied to the dry kibbles to complete the test composition.

Each test composition was compared against a control composition in a palatability test. The control compositions were prepared by topically applying the commercial liquid palatant enhancer (0.5%, dry weight basis) to the dry, commercial cat food (Feline w/d) after sequential application of water (1.5%), soy bean oil (1.7%, dry weight basis) and choice white grease (1.0%, dry weight basis) followed by the commercial dry palatant enhancer (1.4% dry weight basis). Palatability was determined by comparing the test composition and the control composition in a standard two-bowl preference test over two days with 20 cats. Results are shown in Table 5 below.

TABLE 5
Results for Example 5

| | 2,5-dimethyl-4- | | | | |
|---|---|---|---|---|---|
| | hydroxy- | | | | |
| | 3(2H)-furanone | | | | |
| | in liquid palatant | | | | Statistical |
| Exp. | enhancer (ppm, | Status (vs. | Intake | Preference | Signifi- |
| No. | dry wt. of kibble) | control) | Ratio | (%) | cance |
| 1 | 5.0 | Win | 0.7676 | 92.0/8.0 | 0.0001 |
| 2 | 10.0 | Win | 0.8135 | 88.0/8.0 | 0.0001 |
| 3 | 20.0 | Win | 0.8151 | 88.0/12.0 | 0.0001 |
| 4 | 30.0 | Win | 0.7546 | 80.0/20.0 | 0.0001 |
| 5 | 40.0 | Win | 0.7626 | 88.0/12.0 | 0.0001 |
| 6 | 50.0 | Win | 0.8505 | 92.0/8.0 | 0.0001 |

Second set of experiments

| | | | | | |
|---|---|---|---|---|---|
| 7 | 5.0 | Win | 0.9385 | 100.0/0.0 | 0.0001 |
| 8 | 10.0 | Win | 0.7990 | 84.0/12.0 | 0.0001 |
| 9 | 20.0 | Win | 0.8128 | 92.0/8.0 | 0.0001 |
| 10 | 30.0 | Win | 0.8912 | 92.0/4.0 | 0.0001 |
| 11 | 40.0 | Win | 0.8012 | 80.0/16.0 | 0.0001 |
| 12 | 50.0 | Win | 0.8339 | 87.5/12.5 | 0.0001 |

### Example 6

This example demonstrates the effect of 2,5-dimethyl-4-hydroxy-3(2H)-furanone as a palatability enhancer for a dry, commercial cat food composition. The experiment comprised mixing various amounts of 2,5-dimethyl-4-hydroxy-3(2H)-furanone with a commercial liquid palatant enhancer as a carrier to form a test flavor. Each of the test flavors (0.5%, dry weight basis) was topically applied to a dry, commercial cat food (Feline w/d from Hill's Pet Nutrition, Inc.) after sequential application of water (1.5%), soy bean oil (1.7%, dry weight basis) and choice white grease (1.0%, dry weight basis). Finally, a commercial dry palatant enhancer (1.4% dry weight basis) was topically applied to the dry kibbles to complete the test composition.

Each test composition was compared against a control composition in a palatability test. The control compositions were prepared by topically applying the commercial liquid palatant enhancer (0.5%, dry weight basis) to the dry, commercial cat food (Feline w/d) after sequential application of water (1.5%), soy bean oil (1.7%, dry weight basis) and choice white grease (1.0%, dry weight basis) followed by the commercial dry palatant enhancer (1.4% dry weight basis). Palatability was determined by comparing the test composition and the control composition in a standard two-bowl preference test over two days with 20 cats. Results are shown in Table 6 below.

TABLE 6
Results for Example 6

| | 2,5-dimethyl-4- | | | | |
|---|---|---|---|---|---|
| | hydroxy- | | | | |
| | 3(2H)-furanone | | | | |
| | in liquid palatant | | | | Statistical |
| Exp. | enhancer (ppm, | Status | Intake | Preference | Signifi- |
| No. | dry wt. of kibble) | (vs. control) | Ratio | (%) | cance |
| 1 | 1 | Win | 0.7653 | 80.0/20.0 | 0.0004 |
| 2 | 2 | Win | 0.8280 | 82.6/17.6 | 0.0001 |
| 3 | 3 | Win | 0.9457 | 95.7/4.3 | 0.0001 |
| 4 | 4 | Win | 0.8426 | 84.0/16.0 | 0.0001 |
| 5 | 5 | Win | 0.8957 | 88.9/11.1 | 0.0001 |
| 6 | 10 | Win | 0.8596 | 95.8/4.2 | 0.0001 |

Second set of experiments

| | | | | | |
|---|---|---|---|---|---|
| 7 | 1 | Win | 0.9077 | 95.8/4.2 | 0.0001 |
| 8 | 2 | Win | 0.7613 | 84.0/16.0 | 0.0006 |
| 9 | 3 | Win | 0.8206 | 86.4/13.6 | 0.0001 |
| 10 | 4 | Win | 0.8957 | 95.7/4.2 | 0.0001 |
| 11 | 5 | Win | 0.8596 | 87.5/12.5 | 0.0001 |
| 12 | 10 | Win | 0.7976 | 85.0/15.0 | 0.0001 |

### Example 7

This example demonstrates the effect of 5-methyl-2-ethyl-4-hydroxy-3(2H)-furanone as a palatability enhancer for a dry, commercial cat food composition. The experiment comprised mixing various amounts of 5-methyl-2-ethyl-4-hydroxy-3(2H)-furanone with a commercial liquid palatant enhancer as a carrier to form a test flavor. Each of the test flavors (0.5%, dry weight basis) was topically applied to a dry, commercial cat food (Feline w/d from Hill's Pet Nutrition, Inc.) after sequential application of water (1.5%), soy bean oil (1.7%, dry weight basis) and choice white grease (1.0%, dry weight basis). Finally, a commercial dry palatant enhancer (1.4% dry weight basis) was topically applied to the dry kibbles to complete the test composition.

Each test composition was compared against a control composition in a palatability test. The control compositions were prepared by topically applying the commercial liquid palatant enhancer (0.5%, dry weight basis) to the dry, commercial cat food (Feline w/d) after sequential application of water (1.5%), soy bean oil (1.7%, dry weight basis) and choice white grease (1.0%, dry weight basis) followed by the commercial dry palatant enhancer (1.4% dry weight basis). Palatability was determined by comparing the test composition and the control composition in a standard two-bowl preference test over two days with 20 cats. Results are shown in Table 7 below.

TABLE 7
Results for Example 7

| | 5-methyl-2- | | | | |
|---|---|---|---|---|---|
| | ethyl-4- | | | | |
| | hydroxy- | | | | |
| | 3(2H)-furanone | | | | |
| | in liquid palatant | | | | Statistical |
| Exp. | enhancer (ppm, | Status (vs. | Intake | Preference | Signifi- |
| No. | dry wt. of kibble) | control) | Ratio | (%) | cance |
| 1 | 5 | Win | 0.8254 | 88.0/12.0 | 0.0001 |
| 2 | 20 | Win | 0.9731 | 100.0/0.0 | 0.0015 |

Second set of experiments

| | | | | | |
|---|---|---|---|---|---|
| 1 | 5 | Win | 0.7476 | 78.3/21.7 | 0.0001 |
| 2 | 20 | Win | 0.8679 | 92.0/8.0 | 0.0001 |

All references cited above are incorporated by reference into this patent.

The words “comprise,” “comprises,” and “comprising” are to be interpreted inclusively rather than exclusively.

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

1. A composition for animal consumption comprising a palatability enhancing amount of an alpha cyclic enolone.

2. The composition as set forth in claim 1 wherein the composition comprises from about 0.0001% to about 2% by weight (dry weight basis) of an alpha cyclic enolone compound.

3. The composition as set forth in claim 1 wherein the alpha cyclic enolone is a 2-hydroxy-2-cyclopenten-1-one compound of the formula: 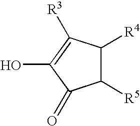
wherein each of R³, R⁴ and R⁵ are independently selected from the group consisting of hydrogen and alkyl.

4. The composition as set forth in claim 3 wherein the alpha cyclic enolone is a 2-hydroxy-2-cycloppenten-1-one compound selected from the group consisting of 3-methyl-2-hydroxy-2-cyclopenten-1-one, 3-ethyl-2-hydroxy-2-cyclopenten-1-one, 3,5-dimethyl-2-hydroxy-2-cyclopenten-1-one, 3,4-dimethyl-2-hydroxy-2-cyclopent-1-one and 3-ethyl-4-methyl-2-hydroxy-2-cyclopenten-1-one or mixtures thereof.

5. The composition as set forth in claim 1 wherein the alpha cyclic enolone is a 4-hydroxy-3(2H)-furanone compound of the formula: 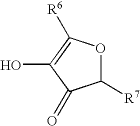
wherein each of R⁶ and R⁷ are independently selected from the group consisting of hydrogen and alkyl.

6. The composition as set forth in claim 5 wherein the alpha cyclic enolone is a 4-hydroxy-3(2H)-furanone compound selected from the group consisting of 5-methyl-4-hydroxy-3(2H)-furanone, 2,5-dimethyl-4-hydroxy-3(2H)-furanone, 5-methyl-2-ethyl-4-hydroxy-3(2H)-furanone, 5-ethyl-2-methyl-4-hydroxy-3(2H)-furanone and 2,5-diethyl-4-hydroxy-3(2H)-furanone or mixtures thereof.

7. The composition as set forth in claim 1 wherein the alpha cyclic enolone is a 3-hydroxy-2(5H)-furanone compound of the formula: 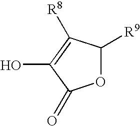
wherein each of R⁸ and R⁹ are independently selected from the group consisting of hydrogen and alkyl.

8. The composition as set forth in claim 7 wherein the alpha cyclic enolone is a 3-hydroxy-2(5H)-furanone compound selected from the group consisting of 4,5-dimethyl-3-hydroxy-2(5H)-furanone and 4-ethyl-5 -methyl-3 -hydroxy-2(5H)-furanone or mixtures thereof.

9. The composition as set forth in claim 1 wherein the alpha cyclic enolone is a 3-hydroxy-4-pyranone compound of the formula: 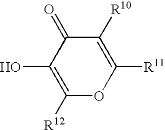
wherein each of R¹⁰, R¹¹ and R¹² are independently selected from the group consisting of hydrogen and alkyl.

10. The composition as set forth in claim 9 wherein the alpha cyclic enolone is a 3-hydroxy-4-pyranone compound selected from the group consisting of 2-methyl-3-hydroxy-4-pyranone and 2-ethyl-3-hydroxy-4-pyranone or mixtures thereof.

11. The composition as set forth in claim 1 wherein:
the composition comprises from about 0.001% to about 1.0% by weight (dry weight basis) of said alpha cyclic enolone compound; and
the composition is manufactured for consumption by a canine.

12. The composition as set forth in claim 11 wherein the composition comprises from about 0.005% to about 0.2% by weight (dry weight basis) of said alpha cyclic enolone compound.

13. The composition as set forth in claim 1 wherein:
the composition comprises from about 0.0001% to about 1.0% by weight (dry weight basis) of said alpha cyclic enolone compound; and
the composition is manufactured for consumption by a feline.

14. The composition as set forth in claim 13 wherein the composition comprises from about 0.0005% to about 0.2% by weight (dry weight basis) of said alpha cyclic enolone compound.

15. The composition as set forth in claim 1 wherein the composition is a nutritional diet, a supplement, an animal treat or toy.

16. A method for enhancing the palatability of a pet food composition, the method comprising adding a palatability enhancing amount of an alpha cyclic enolone compound to a pet food composition.

17. The method as set forth in claim 16, wherein the method comprises adding from about 0.0001% to about 2% by weight (dry weight basis) of an alpha cyclic enolone compound to the food composition.

18. A method for increasing ingestion of a food by an animal, the method comprising feeding the animal a composition of claim 1.

19. A process for preparing a composition for animal consumption, the process comprising topically applying a flavor palatant comprising an alpha cyclic enolone to a food composition.

20. An animal treat, wherein the treat comprises an alpha cyclic enolone compound.

21. An animal treat according to claim 20, wherein the treat comprises from about 0.0001% to about 2% by weight (based on dry weight of the treat) of an alpha cyclic enolone compound.

22. An animal treat according to claim 20, wherein:
the treat comprises from about 0.001% to about 1.0% of an alpha cyclic enolone compound (based on dry weight of the treat), and
the treat is manufactured for consumption by a dog.

23. An animal toy, wherein the toy comprises an alpha cyclic enolone compound.

24. An animal toy according to claim 23, wherein the treat comprises from about 0.0001% to about 2% by weight (based on dry weight of the toy) of an alpha cyclic enolone compound.

25. An animal toy according to claim 23, wherein:
the toy comprises from about 0.001% to about 1.0% of an alpha cyclic enolone compound (based on dry weight of the toy), and
the treat is manufactured for consumption by a dog.
